(19) United States
(12) Patent Application Publication
(10) Pub. No.: US 2008/0009784 A1
(43) Pub. Date: Jan. 10, 2008

(54) Dialysis catheter

(76) Inventors: John D. Leedle, Philadelphia, PA (US); James F. McGuckin JR., Radnor, PA (US)

Correspondence Address: Rex Medical, 1011 High Ridge Road, Stamford, CT, 06905, US

(21) Appl. No.: 11/270,272

(22) Filed: Nov. 9, 2005

Related U.S. Application Data
(60) Provisional application No. 60/629,997, filed on Nov. 22, 2004.

(51) Int. Cl. A61M 3/00

(52) U.S. Cl. 604/43

(57) ABSTRACT

A catheter comprising an inner tube having a distal end, a first lumen and a first delivery opening communicating with the first lumen for delivery of fluid to a patient and a second outer tube having a distal end, a second lumen and a first withdrawal opening communicating with the second lumen to withdraw fluid from the patient. The inner tube is positioned within the outer tube and the inner and outer tubes are relatively movable between a first and second position. In the first position, the distal end of the inner tube is spaced a first distance from the distal end of the outer tube to enable withdrawal of fluid and in the second position the withdrawal opening is blocked to prevent withdrawal of fluid from the patient.

This application claims priority from provisional application No. 60/629,997, filed Nov. 22, 2004.

### BACKGROUND

1. Technical Field

This application relates to a catheter and more particularly to a multi-lumen catheter which facilitates hemodialysis.

2. Background of Related Art

Hemodialysis is a well known method of providing renal (kidney) function by circulating blood. The kidneys are organs which function to extract water and urea, mineral salts, toxins, and other waste products from the blood with filtering units called nephrons. From the nephrons the collected waste is sent to the bladder for excretion. For patients having one or both defective kidneys, the hemodialysis procedure is life saving because it provides a machine to simulate the function of the kidneys.

In the hemodialysis procedure, blood is withdrawn from the patient's body through a catheter or tube and transported to a dialysis machine, also commonly referred to as a kidney machine. In the dialysis machine, toxins and other waste products diffuse through a semi-permeable membrane into a dialysis fluid closely matching the chemical composition of the blood. The filtered blood, i.e. with the waste products removed, is then returned to the patient's body. The dialysis catheters have one or more outflow lumens to withdraw the blood from the patient and one or more inflow lumens to transport the filtered blood to the patient. The dialysis catheter is typically inserted through the jugular vein and maneuvered into position through the superior vena cava into the right atrium to provide high blood flow. As can be appreciated, proper access to the patient's blood and transport of the blood to and from the dialysis machine for this extended period of time is critical to hemodialysis.

The dialysis catheter can be positioned in the patient for several months or even years. The patient would then typically undergo the dialysis procedure several times a week. In current catheters, the catheter lumens remain open between dialysis sessions. This can cause blood clotting in the lumens which can affect blood flow through the catheter during dialysis. Additionally, between dialysis procedures, the catheter is flushed with heparinized saline. However, since the catheter lumen is open, blood can enter at the distal end of the lumen to mix with heparin. Consequently, there could be a loss of heparin lock which can cause blood clots at the catheter tip and can adversely affect blood flow through the catheter during dialysis. Therefore, it would be advantageous to provide a catheter which could reduce the formation of blood clots in the catheter lumens between dialysis sessions and could reduce the chances of loss of heparin lock.

Fibrin sheath growth around the outside of the catheter also occurs since dialysis catheters, as noted above, are oftentimes implanted for several months or even years. This fibrin growth, caused by the body's attempt to reject the catheter as a foreign body, could result in blocking of the holes in the sidewalls of the catheter. These sidewall holes communicate with the catheter lumens for delivery or withdrawal of blood and therefore blocking of the holes can adversely affect blood flow. It would therefore also be advantageous to provide a catheter which could disrupt fibrin sheath to reduce the likelihood of blocking of the catheter holes.

### SUMMARY

The present invention overcomes the problems and deficiencies of the prior art. The present invention provides a dialysis catheter comprising an inner tube having a distal end, a first lumen and a first delivery opening communicating with the first lumen for delivery of fluid to a patient and a second outer tube having a distal end, a second lumen and a first withdrawal opening communicating with the second lumen to withdraw fluid from a patient. The inner tube is positioned within the outer tube and the inner and outer tubes are relatively movable between a first and second position. In the first position, the distal end of the inner tube is spaced a first distance from the distal end of the outer tube to enable withdrawal of fluid and in the second position the withdrawal opening is blocked to prevent withdrawal of fluid from the patient.

In one embodiment, the inner tube is positioned substantially centrally within the outer tube. In one embodiment, the delivery opening is positioned in a sidewall of the inner tube. A second delivery opening can optionally be positioned in the sidewall of the inner tube.

In one embodiment, the distal end of the inner tube could be closed to close off the distal end of the first lumen so that in the second position, the delivery opening is also blocked. In another embodiment, the first lumen can terminate in the first delivery opening and can be dimensioned to receive a guidewire for over the wire insertion of the catheter.

In one embodiment, the inner tube includes a ledge and is withdrawn proximally until the ledge contacts a portion of the outer tube to block the withdrawal opening such that the ledge acts as a stop for proximal movement of the inner tube. In an alternate embodiment, the outer tube is advanced distally to block the withdrawal lumen as a head portion of the inner tube blocks the withdrawal opening.

The inner tube can be provided with a seal to prevent the egress of fluid.

The present invention also provides a method for performing dialysis comprising:
a) providing a dialysis catheter having an inner tube with a first lumen and an outer tube with a second lumen, the inner tube positioned within the outer tube;
b) inserting the catheter into a patient's body;
c) initiating a first dialysis mode so blood is delivered to a patient through one lumen and blood is withdrawn from the patient through the other lumen;
d) after completion of the first dialysis mode moving one or both of the inner and outer tubes to effectively block one of the lumens to prevent the entry of blood into the lumen; and
e) subsequently moving one or both of the inner and outer tubes to unblock the lumen to enable initiation of a second dialysis mode.

In one embodiment, movement of one or both of the inner and outer tubes further blocks both lumens. In one embodiment, the step of initiating a first dialysis mode to deliver blood includes the step of providing blood through at least one opening in a sidewall of the inner tube.

The method may include the step of inserting the catheter over a guidewire with the catheter extending through a subcutaneous tissue tunnel. The method may also include inserting a stiffening member through the catheter such that the stiffening member and catheter are together inserted over the guidewire.

### BRIEF DESCRIPTION OF THE DRAWINGS

Preferred embodiment(s) of the present disclosure are described herein with reference to the drawings wherein:

FIG. 1 is a perspective view of a first embodiment of the dialysis catheter of the present invention;

FIG. 2 is an enlarged broken perspective view of the distal end portion of the catheter of FIG. 1;

FIG. 3 is a longitudinal cross-sectional view of the distal end portion of FIG. 2 showing the inner tube in the retracted position between dialysis procedures (modes);

FIG. 4A is a transverse cross-sectional view taken along line 4-4 of FIG. 3;

FIG. 4B is a transverse cross-sectional view similar to FIG. 4A except showing an alternate embodiment of the catheter lacking transverse struts engaging the inner tube;

FIG. 5 is an enlarged view in partial cross-section of the hub portion of the catheter of FIG. 1 depicting the direction of movement of the inner tube to the extended position for dialysis;

FIG. 5A is a cross-sectional view similar to FIG. 3 showing the movement of the inner tube from a retracted position (shown in phantom) between dialysis sessions to the extended (advanced) position for performing a dialysis procedure;

FIG. 6 is a view similar to FIG. 5 except showing an alternate embodiment of the dialysis catheter of the present invention wherein the outer tube moves, the drawing depicting the direction of movement of the outer tube to the retracted position;

FIG. 6A is a longitudinal cross-sectional view of the distal end portion of the catheter of FIG. 6 showing movement of the outer tube from an extended (advanced) position for performing dialysis to a retracted position (shown in phantom) between dialysis procedures;

FIG. 6B is a view similar to FIG. 1 except showing a perspective view of the dialysis catheter of FIG. 6;

FIG. 6C is a close up view of the cuff region of the dialysis catheter of FIG. 6B;

FIG. 7 is a longitudinal cross-sectional view of the distal end portion of another alternate embodiment of the dialysis catheter of the present invention having an inner tube with a closed distal end and showing the inner tube in the retracted position between dialysis procedures;

FIG. 8 is a cross-sectional view similar to FIG. 7 showing the inner tube in the extended (advanced) position for performing a dialysis procedure;

FIG. 9 is a longitudinal cross-sectional view of the distal end portion of another alternate embodiment of the dialysis catheter of the present invention showing the inner tube in the retracted position between dialysis procedures;

FIG. 10 is a cross-sectional view similar to FIG. 9 showing the inner tube in the extended (advanced) position for performing a dialysis procedure;

FIGS. 11-13 illustrate one method of inserting the dialysis catheter of FIG. 1 wherein FIG. 11 illustrates the catheter being inserted through the subcutaneous tissue tunnel for advancement over the guidewire loop, FIG. 12 illustrates the catheter in place through the subcutaneous tissue tunnel and forming a loop corresponding to the guidewire loop, and FIG. 13 illustrates the catheter in place extending through the subcutaneous tissue tunnel and into the right internal jugular vein, superior vena cava and right atrium;

FIG. 14 illustrates an alternate method of inserting the dialysis catheter of FIG. 1 without formation of a guidewire loop, the drawing illustrating the guidewire extending through the subcutaneous tissue tunnel and into the right atrium;

FIG. 15 illustrates the catheter in place (prior to removal of the stiffener) extending through the subcutaneous tissue tunnel and into the right internal jugular vein, superior vena cava, and right atrium;

FIG. 16 is a view similar to FIG. 5 except showing an alternate embodiment of a seal for the inner tube;

FIG. 17 is a detailed view of the seal region of FIG. 16 showing the inner tube in the retracted position between dialysis procedures;

FIG. 18 is a detailed view of the seal region of FIG. 16 showing the inner tube in the extended position for performing a dialysis procedure;

FIG. 19 is a view similar to FIG. 5 except showing another alternate embodiment of a seal for the inner tube; and

FIG. 20 is a detailed view of the seal region of FIG. 19 showing the inner tube in the retracted position.

### DETAILED DESCRIPTION OF PREFERRED EMBODIMENTS

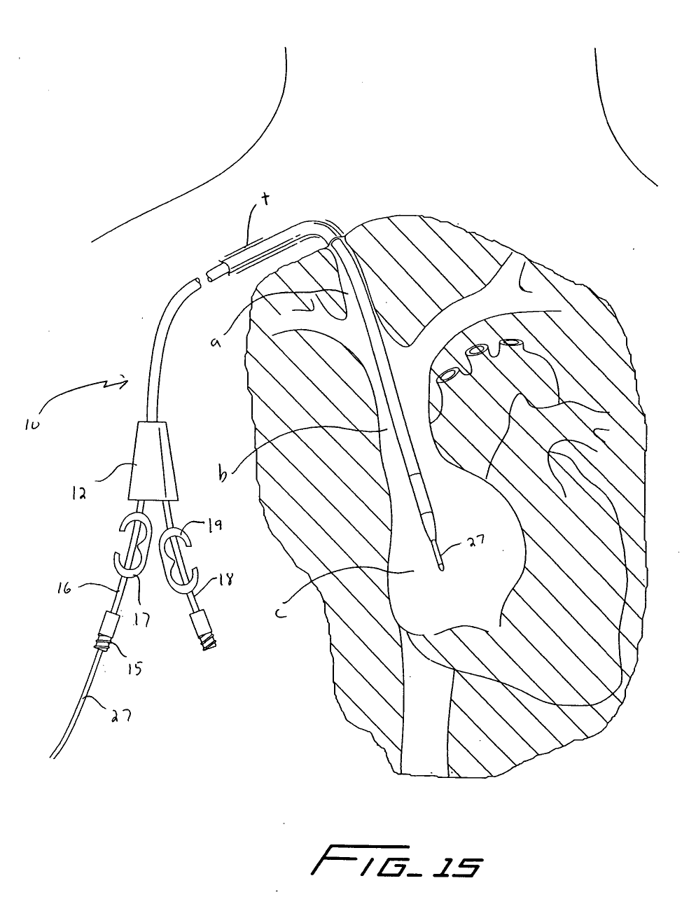

Referring now in detail to the drawings where like reference numerals identify similar or like components throughout the several views, the first embodiment of the catheter of the present invention is designated generally by reference numeral 10. The catheter 10 is typically inserted into an area of high velocity blood flow to ensure sufficient blood can be transported from the body for dialysis. FIG. 15 illustrates the catheter 10 inserted through the right internal jugular vein “a”, into the superior vena cava “b”, and into the right atrium “c”. The catheter 10 can alternately be inserted (not shown) into the left internal jugular vein, into the superior vena cava and into the right atrium “c”. Insertion into the right atrium, from either the right or left side provides the necessary high blood flow to the dialysis machine. The catheter can also be inserted into other areas of the body and through other access sites. Note that the catheter is sufficiently flexible to enable it to bend to accommodate the anatomical curves as shown.

As is well known in the art, the dialysis machine essentially functions as a kidney for patients suffering from kidney failure. Blood is removed from the patient and transported to the dialysis machine where toxins are removed by diffusion through a semi-permeable membrane into a dialysis fluid. The filtered blood is then returned through the catheter body to the patient. As used herein, the terms “inflow” and “outflow” refer to the direction of blood flow with respect to the catheter such that “return”, “delivery” or “venous flow” refers to flow from the dialysis machine to the patient and delivered to the body while “intake”, “withdrawal” or “arterial flow” refers to flow withdrawn from the patient's body and transported to the dialysis machine.

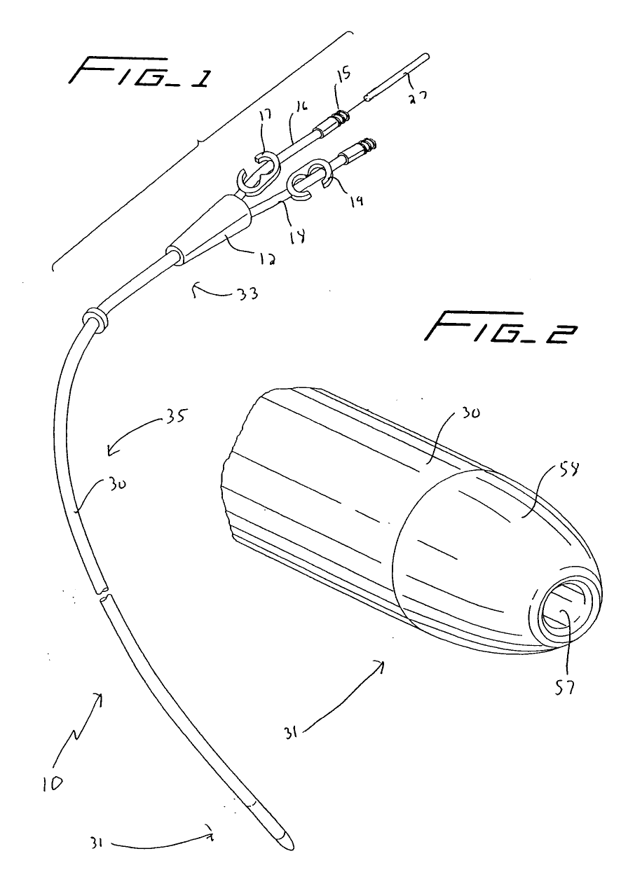

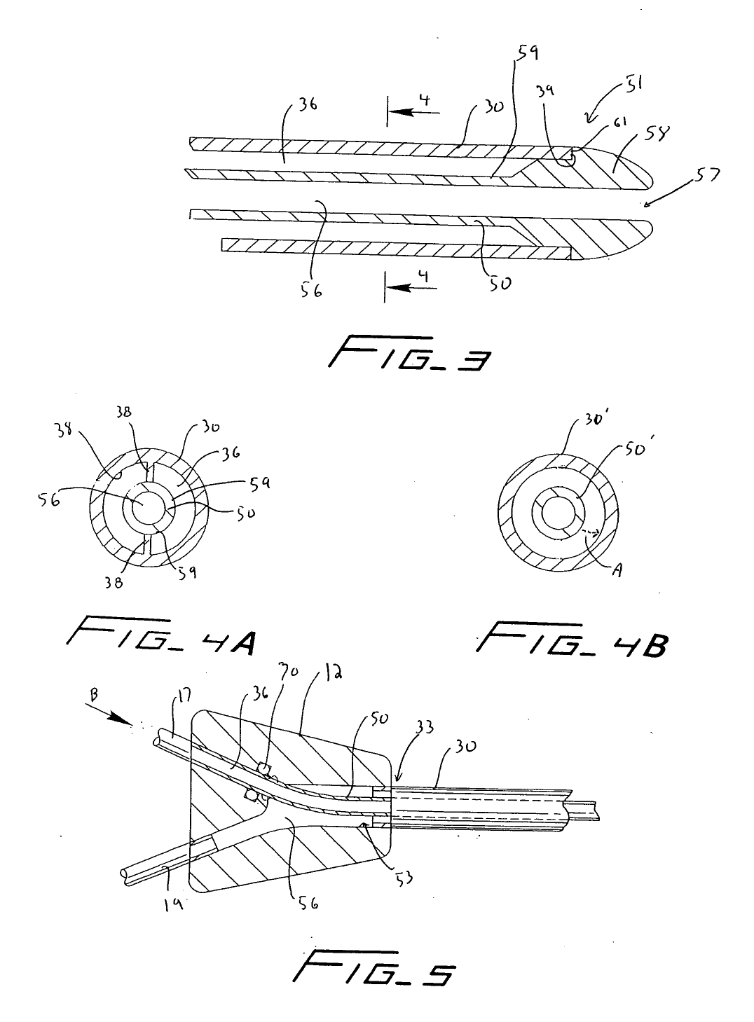

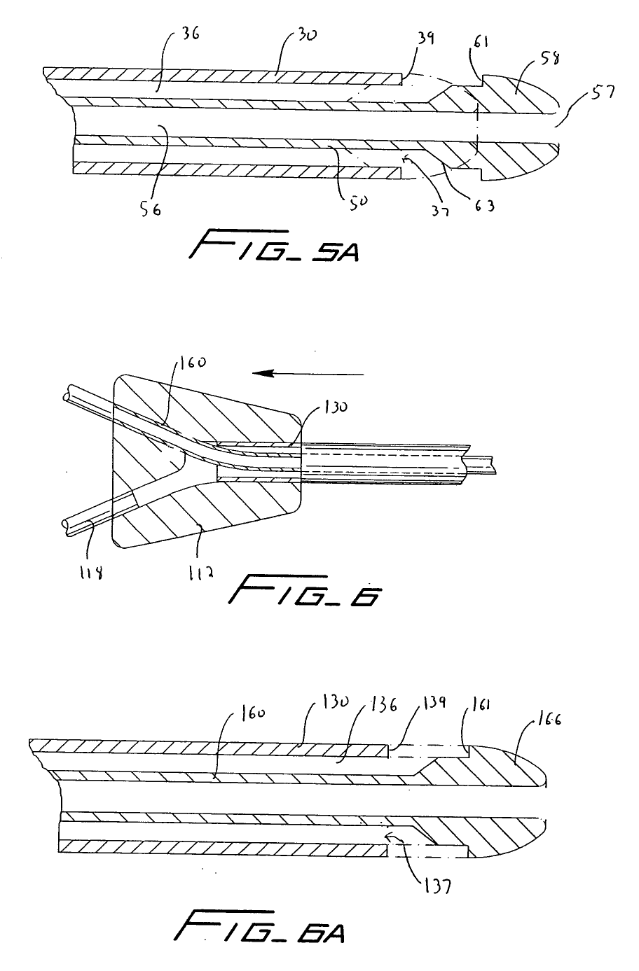

Turning now to FIGS. 1-5, a first embodiment of the catheter of the present invention is illustrated. Catheter 10 has an outer tube 30 and an inner tube 50 positioned therein. Inner tube 50 is preferably disposed substantially centrally within the outer tube 30. Transverse ribs or septum 38, shown in FIG. 4A can be provided in one or more locations along the outer tube 30 of the catheter 10, extending radially inwardly to frictionally engage the outer wall 59 of inner tube 50 (while still enabling sliding movement) and help maintain the centered position of the inner tube 50. FIG. 4B shows an alternate embodiment not having transverse ribs so that the inner tube 50′ is not maintained in a central position but can move radially within outer tube 30′ (see e.g., arrow A). The sliding movement of the tube enables selective opening and blocking of the withdrawal lumen as described in more detail below as the distance between the distal ends of the tubes is varied.

Outer tube 50 has a distal end portion 31, a proximal end portion 33, and an intermediate portion 35. Arterial lumen 36 extends through outer tube 30, from the proximal end to distal end. Since inner tube 50 is positioned in lumen 36, the annular space between the inner wall 38 of the outer tube 30 and the outer wall 59 of the inner tube 50 forms the annular lumen 36 for blood passage, and preferably for blood withdrawal. This arterial (withdrawal) lumen 36 extends longitudinally along the length of the catheter tube 30 and terminates in withdrawal opening 37. Inner tube 50 has a distal end portion 51, a proximal end portion 53, an intermediate portion therebetween, and a venous (delivery) lumen 56 extending therethrough. Opening 57 at the distal end of inner tube 50 is longitudinally aligned with venous lumen 56 so that lumen 56 terminates in delivery opening 57. Proximal end portions 33 and 53 of tubes 30, 50 respectively, extend into hub 12, where the lumens 36, 56 formed in the tubes 30, 50, respectively, are fluidly connected to the respective inflow and outflow tubes 16, 18 to enable return and withdrawal of blood for dialysis. Conventional tube clamps 17 and 19 cut off blood flow through inflow and outflow tubes 16, 18 as desired.

As shown in FIG. 15, an intermediate portion of catheter 10 extends through subcutaneous tissue tunnel “t”, and curves downwardly toward the target site, e.g. the right atrium “c”. This tunnel “t” secures the catheter in place for dialysis for a period of weeks, months, or even years, with a conventional fibrous cuff (not shown) enabling tissue ingrowth over a period of time to enhance retention. The formation of the tunnel “t” and the insertion of the catheter 10 therethrough will be discussed below in conjunction with the discussion of the catheter insertion methods.

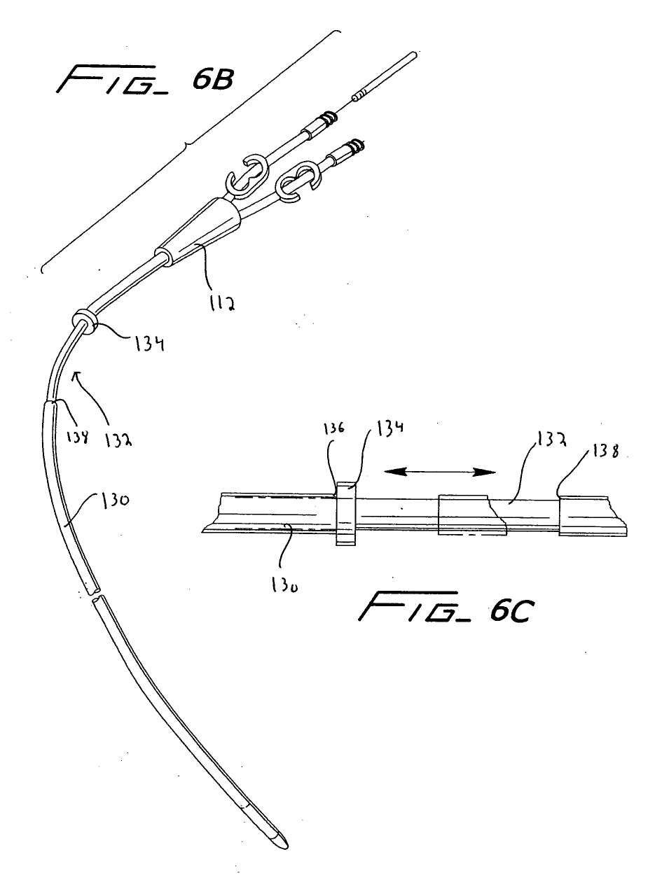

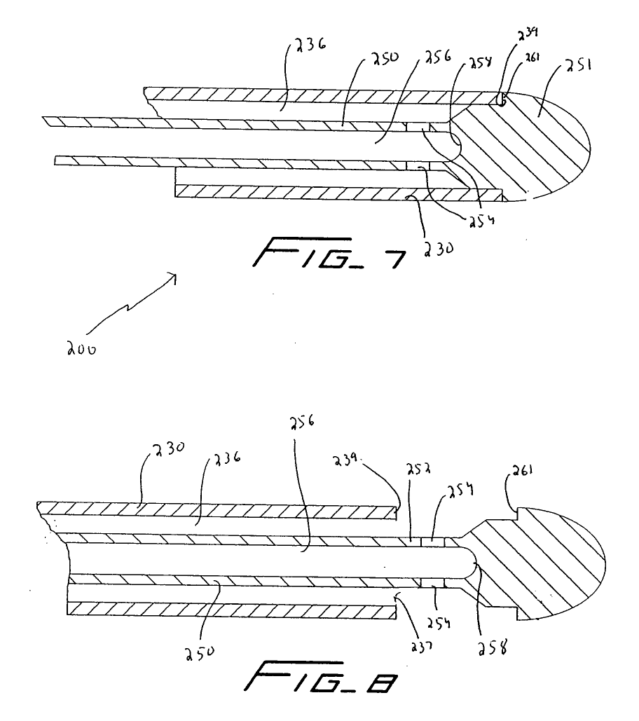

Referring back to FIGS. 1-5, inner tube 50 is slidably positioned within the outer tube 30 for movement between the retracted position of FIG. 3 (and shown in phantom in FIG. 5A) and the extended (advanced) operative position of FIG. 5A spaced a further distance from the edge 39 of the outer tube 30. Arrow B of FIG. 5 shows the direction of movement of the inner tube 50 to its extended position. Inner tube 50, slidably supported by washer 70 which forms a seal, slides within hub 12 and outer tube 30. Since inflow tube 16 is attached to inner tube 50, the user can grasp inflow tube 16 and slide it in the direction of the arrow of FIG. 5 to advance the inner tube 50. Securement means (not shown) could optionally also be provided to retain the inner tube 50 in the retracted position and/or extended position. It is also contemplated that alternatively the outer tube could be slidable with respect to the inner tube. This is shown for example in the embodiment of FIGS. 6 and 6A. Inner tube 160 remains stationary as outer tube 130 moves between an extended position for dialysis and a retracted position (shown in phantom) between dialysis sessions (modes). Also contemplated is both the inner tube and outer tube moving. “Relative movement” as used herein encompasses each of these alternatives.

In the embodiment of FIG. 6 where the outer tube moves, provision could be made, such as by providing a reduced diameter region, to enable the outer tube to slide with respect to the fixed cuff. The larger diameter regions could act as a stop when contacting the cuff. This is illustrated in FIGS. 6B and 6C. Reduced diameter region 132 of tube 130 slides within the opening of cuff 134 when the outer tube 130 is advanced and retracted. In this manner, the tissue ingrowth around the cuff is maintained to maintain securement of the catheter while the fibrin sheath growth around the catheter can be disrupted by the outer tube 130. Edges 136, 138 act as forward and rearward stops.

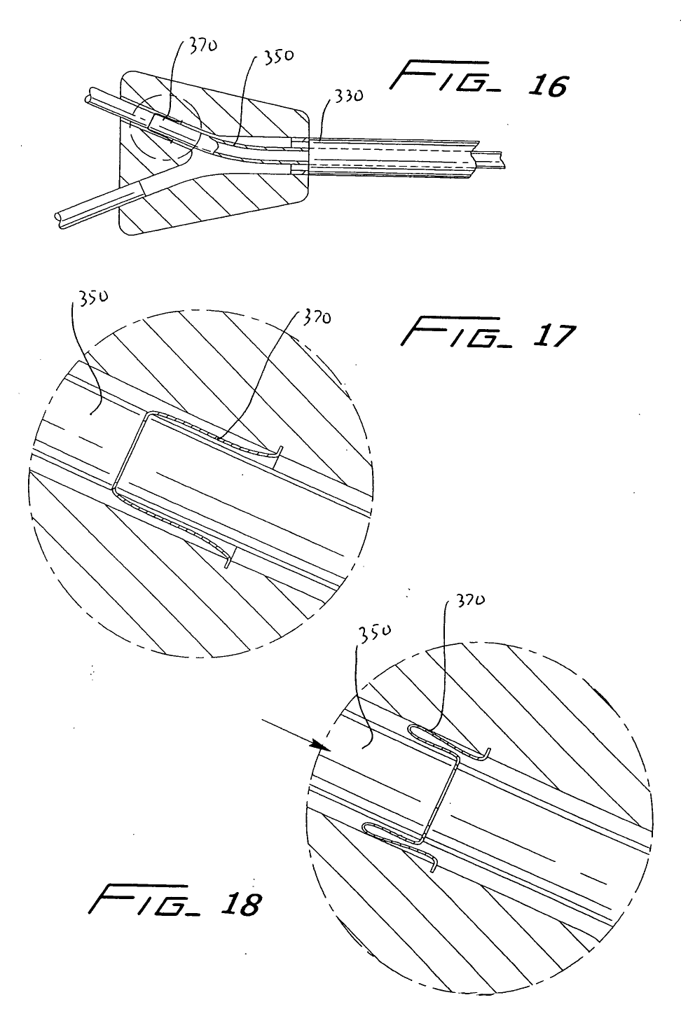

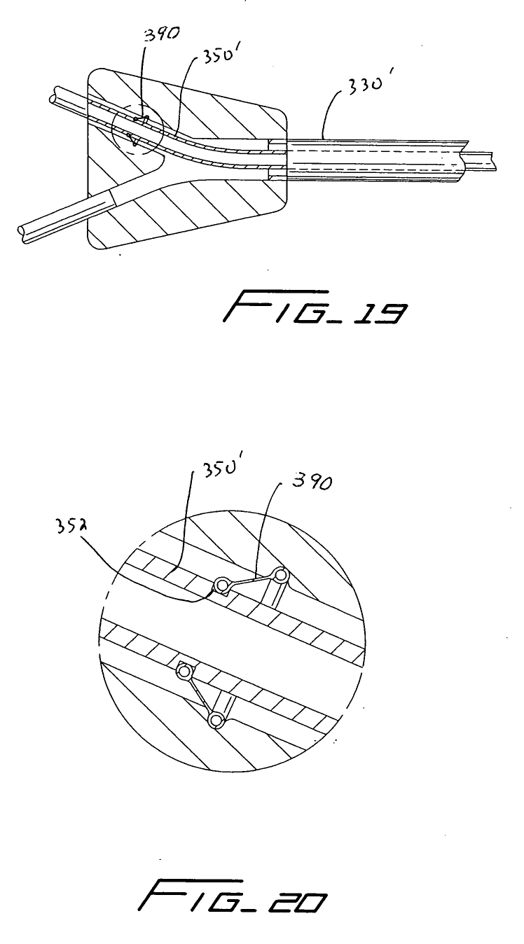

As an alternative to the washer or O-ring seal, a rolling diaphragm seal could be provided as shown in the alternate embodiment of FIGS. 16-18. When the inner tube 350 is advanced (within outer tube 330) from the position of FIG. 17 to the position of FIG. 18, the seal 370 is inverted as shown. This helps to frictionally retain the inner tube. As shown, seal 370 is attached to the outer tube 130. A toggling diaphragm seal is shown in the alternate embodiment of FIGS. 19 and 20. Upon advancement of the inner tube 350′ within outer tube 330′, seal 390 toggles from the position of FIG. 20. This also provides frictional retention of the inner tube 350′. Slot 352 in the inner tube 350′ cooperates with seal 390 to provide additional retention of outer tube 330′ in the advanced and retracted and/or extended positions. Thus, these diaphragm seals prevent the egress of fluid as well as help retain the inner tube in its retracted position between dialysis sessions.

Bumps, projections or locking detents could also be provided on the movable tube which contact the seal or other stationary part of the catheter to help retain the tube in position. Application of sufficient force would enable overriding the force of the bump or detents to slide the tube. This could be achieved manually. Alternatively, a syringe with fluid such as heparinzed saline would be attached to the back end of the catheter to pressurize the inner tube to advance it (in a piston like manner) to overcome the frictional engagement of the inner tube.

Referring back to FIGS. 3 and 5A, nose 58 of inner tube 50 can be integral as shown or a separate piece attached to the distal end of the tube 50. The lumen 56 extending through the inner tube 50 enables blood passage therethrough, and preferably blood delivery to the patient thereby forming a venous delivery lumen. The inner tube 50 can also include one or more side venous (delivery) openings (not shown) formed through the outer wall 59 in fluid communication with lumen 56, also functioning to return blood to the patient's body. Such openings in the sidewall would preferably be formed in a distal location where they could be blocked by the outer tube 30 when the inner tube 50 is in the retracted position of FIG. 3.

Inner lumen 56 of outer tube 50 is also preferably dimensioned to receive a stiffening member in the form of a stiffening rod disclosed in co-pending commonly assigned patent application Ser. No. 10/279,468 filed Oct. 24, 2002, the entire contents of which are incorporated herein by reference. One example of a stiffening member which can be utilized is depicted in part in FIGS. 1 and 15. The stiffening member 27 is inserted into lumen 56 of inner tube 50 and torqued to stiffen the flexible catheter for ease in over the wire insertion and navigation through the small vessels. It is preferably attached to the hub via a proximally located screw thread (not shown) attached to thread 15 of venous extension tubing 17. After placement of the catheter 10, the stiffening member 27 is removed.

Nose 58 of inner tube 50, in the embodiment of FIGS. 1-5, has a stepped portion or ledge 61 which contacts (abuts) the edge 39 of outer tube 30 when the inner tube 50 is in the retracted position, thus acting as a stop. This retraction of inner tube 50 provides a seal for the arterial lumen 36 of outer tube 30 by preventing blood inflow through arterial lumen 36 as it closes off opening 37. Tapered wall 63 functions as a lead in to facilitate withdrawal of the inner tube 50 into outer tube 30. When advanced to the position of FIG. 5A, lumen 36 is reopened for blood inflow for dialysis or other medical procedures as ledge 61 becomes spaced a sufficient distance from edge 39 to expose withdrawal opening 37.

In the alternate embodiment of FIG. 6, inner tube 160 remains stationary and outer tube 130 is retracted in the direction of the arrow to expose opening 137 of lumen 136 to enable blood withdrawal for dialysis or other procedures. When outer tube 130 is moved in the opposite direction to the advanced (extended) position (shown in phantom in FIG. 6A), edge 139 abuts ledge 161 of inner tube 160, thus closing off (sealing) withdrawal opening 137 to prevent egress of blood into arterial lumen 136. Note that outer tube 130 slides within hub 112 (by movement of outflow tubing 118). Securement means (not shown) to retain the outer tube 130 in the retracted and/or advanced positions could be provided.

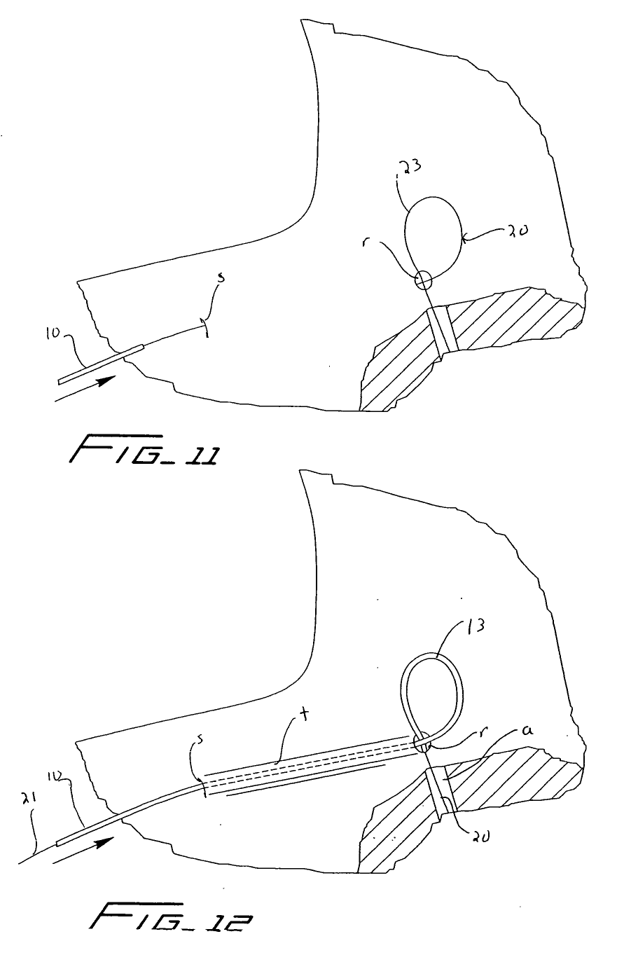

In the alternate embodiment of FIGS. 9 and 10, instead of a stepped portion, inner tube 190 of catheter 170 has a curved surface 192 which mates with angled surface 182 of the outer tube 180 to block blood flow through withdrawal opening 187 of arterial lumen 186 when inner tube 190 is in the retracted position. In the advanced position for dialysis (FIG. 10), lumen 186 is open as withdrawal opening 187 is exposed. Lumen 196 in inner tube 190 provides for blood inflow through delivery opening 197.

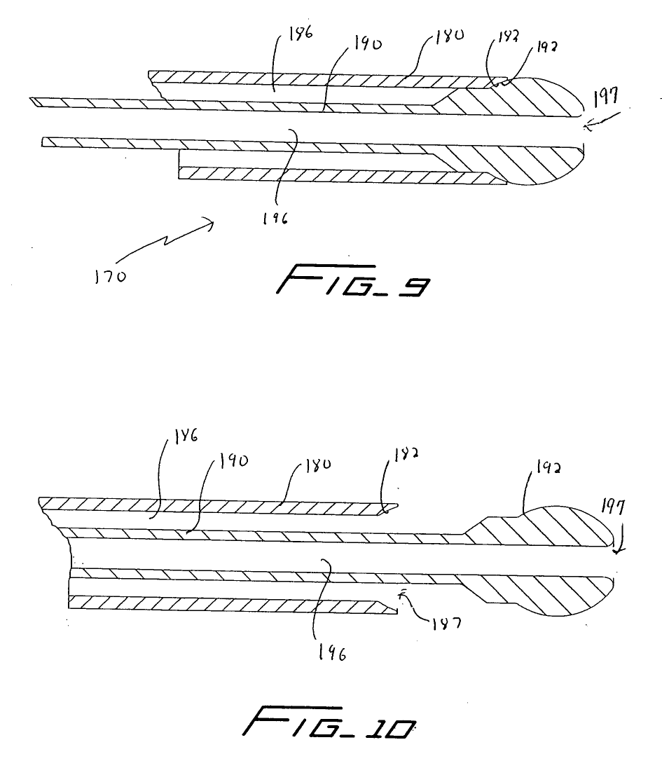

FIGS. 7 and 8 illustrate an alternate embodiment of the dialysis catheter wherein the inner tube lumen is also sealed when the inner tube is in the retracted position between dialysis sessions. More specifically, catheter 200 has an inner tube 250 slidably positioned within outer tube 230. Inner tube 250 has a solid nose 261 forming a closed distal head so that the distal end of venous (delivery) lumen 256 is blocked by wall 258. Blood inflow occurs through delivery openings 254 in sidewall 252 of inner tube 250 which are in fluid communication with lumen 256. When inner tube 250 is withdrawn to its retracted position, as shown in FIG. 7, ledge 261 abuts edge 239 of outer tube 230, thus sealing withdrawal opening 237 of arterial lumen 236. Additionally, withdrawal of the inner tube 250 withdraws sidewall openings 254 inside outer tube 230 to effectively seal or block openings 254 to prevent fluid communication of venous lumen 256 with the vessel. Note, alternatively, the outer tube or both tubes could move to achieve the same function of blocking both the withdrawal and delivery openings 237, 254, respectively.

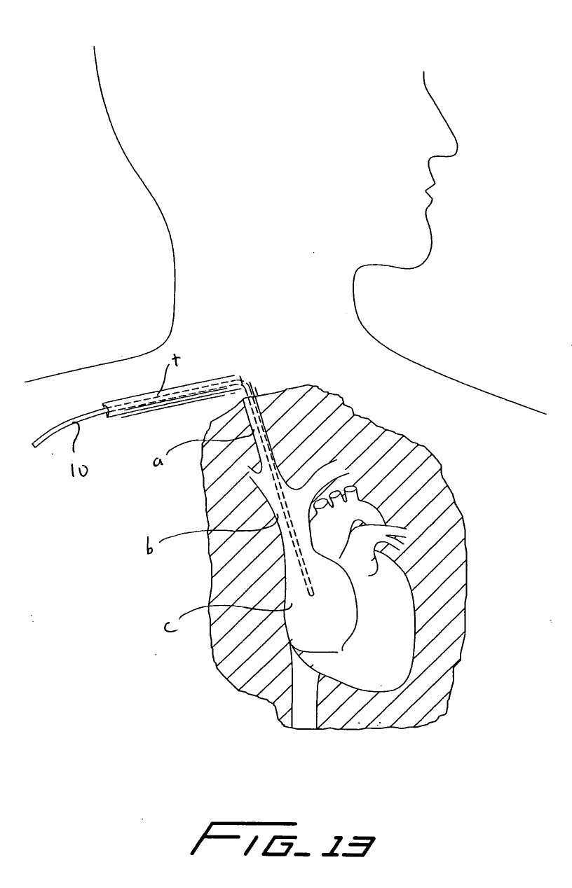

One method of insertion of the catheter of the present invention will now be described in conjunction with FIGS. 11-13. This method provides an entire over the wire system and is described for insertion of catheter 10 of FIG. 1 for use in a dialysis procedure. Although the method will be described for inserting catheter 10, it should be appreciated that the aforedescribed catheters of FIGS. 6 and 9 can be inserted in the same manner. The complete over the wire system is achieved by retraction of the guidewire through the subcutaneous tissue tunnel. One way to retract the guidewire is by the provision of a trocar (not shown) which grasps the guidewire and retracts it through the tissue tunnel and is illustrated and described in detail in pending application Ser. No. 10/279,468.

In this method, after a needle is inserted into the internal jugular vein to properly locate the vessel, a guidewire 20 is inserted through the needle into the right internal jugular vein and into the superior vena cava through incision “r”. The guidewire 20 is further advanced into the right atrium, and preferably into the inferior vena cava. The needle is then withdrawn, leaving the guidewire 20 in place, extending out of the patient's body. Next, a trocar or other guidewire retrieval device (not shown) is inserted through a first incision “s” in the patient, bluntly dissecting and tunneling under the skin, and forced out of the tissue at a second incision or site at the needle/guidewire insertion site, creating a subcutaneous tunnel “t” under the tissue. This tunnel provides a way to secure the catheter. Guidewire 20 is then threaded through a lumen of the trocar or pulled back through the tunnel by a retrieval instrument so it emerges out of first incision “s” and proximal portion 21 extends outside the patient (see FIG. 11). The trocar or retrieval instrument is then withdrawn from the body leaving the guidewire 20 in place as shown. Thus, guidewire 20 extends from the right atrium and superior vena cava, out through the right internal jugular vein and through the tissue tunnel “t”, exiting incision “s”. A loop 23 is maintained in the guidewire outside incision “r”.

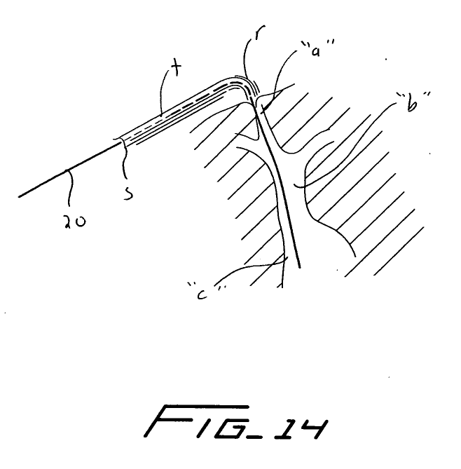

The catheter 10 is then advanced over the guidewire 20 (FIG. 12), through the tissue tunnel, and exiting under the tissue adjacent incision site “r” into the internal jugular vein “a”. The catheter 10, as shown, is formed into a loop 13, tracking the loop 23 of guidewire 20, and then advanced downwardly through the internal jugular vein “a”, the superior vena cava “b” and into the right atrium “c” (FIG. 13). Thus, catheter 10 is threaded over the guidewire, with the proximal portion 21 of the guidewire inserted through the distal opening of lumen 56 of the inner tube 50, through the length of the lumen 56, and through the hub 12 into the inflow tube 16 and out through fitting 15. The catheter 10 is advanced over the guidewire 20, through the tissue tunnel “t” where cuff (not shown) is positioned in the tissue tunnel “t” to aid in securement of the catheter. The guidewire 20 is then withdrawn and the catheter is pushed downwardly and/or pulled back to straighten the loop to position the catheter as shown in FIG. 13.

Note the stiffening member 27 is preferably utilized, i.e. inserted over the guidewire 20 through the fitting 15, inflow tube 16, hub 12, and lumen 56 of inner tube 50 to help guide the catheter 10 as described above. Thus, the guidewire 20 would extend through the lumen 56 of catheter 10 by extending through the central lumen of the stiffening member 27 which is positioned within the lumen 56 of the catheter as shown in FIG. 15. The stiffening member 27 would be removed after insertion of the catheter.

As can be appreciated, the catheter can be inserted in a similar fashion through the left internal jugular vein. In this method, the subcutaneous tissue tunnel will be formed on the left side, and the catheter inserted over the guidewire through the tissue tunnel and through the left internal jugular vein or subclavian vein and into the superior vena cava and right atrium in the same way as described for right side insertion. It should be understood that the catheters of FIGS. 6 and 9 can also be inserted in this fashion.

It should be appreciated that formation of the loop in the guidewire and the catheter is optional and the procedure can be performed without the loop. Thus, in this alternate method of insertion, shown in FIG. 14, guidewire 20 is inserted through the tissue tunnel “t” as described above and out through the first incision “s” except without a loop. Catheter 10 would then be threaded over the guidewire through incision “s” and directly through the site adjacent incision site “r” and into the jugular vein “a”.

As can be appreciated, the foregoing methods provide a complete over the wire insertion of the catheter. It is also contemplated that alternatively, the catheter and stiffener can first be inserted through the tunnel, and then the guidewire threaded back through the catheter and stiffener, i.e., through the lumen of the stiffener, hub and fitting. The catheter and stiffener could then be inserted through the superior vena cava into the right atrium. Thus, although not providing for an entire over the wire system, it provides a partial over the wire system which eliminates the need for a tear way introducer sheath and the problems associated with the sheath.

In another method a trocar (not shown) can be attached to the catheter, to assist advancement of the catheter through the tissue tunnel, so it emerges out through the second incision as described in co-pending application ser. No. 10/279,468. The trocar is then detached from the catheter. The catheter is then bent as necessary and threaded over the guidewire into jugular vein, superior vena cava, and right atrium. This also provides a partial over the wire system.

In the embodiment of FIGS. 7 and 8, the catheter does not allow for over the wire insertion, but has the feature of the relative movement of the catheter tubes to seal off the withdrawal lumen between dialysis sessions. It also has the additional feature of sealing the venous delivery lumen as described above.

After insertion of the catheter in any of the foregoing methods so it is placed in the position of FIG. 15, the dialysis procedures can occur. That is, blood withdrawal and delivery through the tubes and lumens is effected to perform a first dialysis mode. After the dialysis session, the catheter remains in the body until the next session. However, the inner tube (and/or outer tube) 13 are moved in the manner described above to close off the withdrawal opening and lumen to prevent blood flow therein. The stop, such as the inner tube ledge described above, limits movement of the tubes. The withdrawal opening remains sealed (or blocked) until the next dialysis session. For the next dialysis procedure (mode), the inner tube (and/or outer tube) is moved to open the withdrawal opening to perform another dialysis mode. After this second dialysis mode, the tube(s) can again be moved to effectively block the opening. Thus, relative movement of the tubes can be selectively repeated the desired number of times to enable opening and blocking the withdrawal opening and lumen. As noted above, in the embodiment of FIGS. 7 and 8, relative movement of the tubes would also effectively open and block fluid access to the venous lumen of the inner tube.

The catheters described above can optionally include a surface treatment on the exterior and/or the interior. The surface treatments can include for example, a hydrophilic coating to increase lubricity and facilitate insertion, a drug coating such as heparin or containing IIb, IIIa inhibitors, inert coating substances such as Sorins carbon coating, and/or active coatings such as a silver ion coating.

It should be appreciated that although the catheter is described herein as a dialysis catheter for hemodialysis, the catheter disclosed herein could have other surgical applications, such as drug delivery, r blood sampling or plasmapheresis.

As noted above, although described as either the inner tube or the outer tube moving, it is also contemplated in each of the embodiments that both tubes could move to provide a way to open and seal off the outer tube lumen. Thus, relative movement to block off blood flow can be achieved by movement of either tube or both tubes.

While the above description contains many specifics, those specifics should not be construed as limitations on the scope of the disclosure, but merely as exemplifications of preferred embodiments thereof. For example, reinforcement such as a coil could be embedded in the inner tube throughout all or part of its length to enhance rigidity, improve pushability and kink resistance, and prevent creep. Those skilled in the art will envision many other possible variations that are within the scope and spirit of the disclosure as defined by the claims appended hereto.

1. A catheter comprising an inner tube having a distal end, a first lumen and a first delivery opening communicating with the first lumen for delivery of fluid to a patient, an outer tube having a distal end, a second lumen and a first withdrawal opening communicating with the second lumen to withdraw fluid from the patient, the inner tube being positioned within the outer tube, the inner and outer tubes being relatively movable between a first and second position, wherein in the first position the distal end of the inner tube is spaced a first distance from the distal end of the outer tube to enable withdrawal of fluid and in the second position the withdrawal opening is blocked to prevent withdrawal of fluid from the patient, the inner tube being advanceable by application of fluid pressure.

2. The catheter of claim 1, wherein the inner tube is positioned substantially centrally within the outer tube.

3. The catheter of claim 1, wherein the delivery opening is positioned in a sidewall of the inner tube.

4. The catheter of claim 3, wherein the distal end of the inner tube is sealed to close off the distal end of the first lumen.

5. The catheter of claim 3, further comprising a second delivery opening formed in a sidewall of the inner tube, the distal end of the inner tube being closed to close off the distal end of the first lumen.

6. The catheter of claim 4, wherein in the second position the delivery opening is blocked.

7. The catheter of claim 1, wherein the first delivery opening is positioned at the distal end of the inner tube longitudinally aligned with the first lumen.

8. The catheter of claim 1, wherein the first lumen terminates in the first delivery opening and the first lumen is dimensioned to receive a guidewire for over the wire insertion of the catheter.

9. The catheter of claim 1, wherein the inner tube includes a ledge, and the inner tube is withdrawn proximally until the ledge contacts a portion of the outer tube to block the withdrawal opening, the ledge providing a stop for proximal movement of the inner tube.

10. The catheter of claim 1, wherein the outer tube is advanced distally to block the withdrawal lumen as a head portion of the inner tube blocks the withdrawal opening.

11. The catheter of claim 10, wherein the outer tube has a reduced diameter region to slide within a tissue ingrowth cuff positioned around the outer tube.

12. The catheter of claim 1, wherein in the second position the delivery opening is blocked.

13. The catheter of claim 1, wherein the inner tube slides within the outer tube and a seal is provided around the inner tube to prevent egress of fluids.

14-19. (canceled)

20. The catheter of claim 2, further comprising a septum extending radially between the outer tube and inner tube to help center the inner tube.

21. The catheter of claim 1, further comprising a stiffening member positioned within the inner tube for insertion of the catheter.

22. The catheter of claim 21, wherein the stiffening member has a lumen for insertion over a guidewire.

23. The catheter of claim 1, wherein the inner tube includes a tapered wall to facilitate withdrawal of the inner tube into the outer tube.

24. The catheter of claim 1, wherein the outer tube has an edge which provides a stop for retraction of the inner tube or advancement of the outer tube.

25. The catheter of claim 1, further comprising a septum extending radially inward from the outer tube to frictionally engage the inner tube, wherein advancement of the inner tube overcomes the frictional engagement of the inner tube.
